(19) United States
(12) Patent Application Publication
(10) Pub. No.: US 2007/0003536 A1
(43) Pub. Date: Jan. 4, 2007

(54) Topical skin compositions, their preparation, and their use

(76) Inventors: Amy C. Zimmerman, Grand Rapids, MI (US); Debra J. Deppa, Grand Rapids, MI (US); Deborah A. O'Toole, Ionia, MI (US); James R. Mayne, Lowell, MI (US)

Correspondence Address: Connie B. Berg Esq.;Alticor, Inc., 7575 Fulton Street East, 78-2G, Ada, MI, 49355, US

(21) Appl. No.: 11/497,152

(22) Filed: Jul. 31, 2006

Related U.S. Application Data
(63) Continuation-in-part of application No. 10/155,305, filed on May 24, 2002.
Application No. 10/155,305 is a continuation-in-part of application No. PCT/US00/31933, filed on Nov. 21, 2000.

(51) Int. Cl. A61K 38/44; A61K 36/752; A61K 36/87; A61K 36/82; A61K 31/7048; A61K 31/353; A61K 31/355; A61K 31/235

(52) U.S. Cl. 424/94.4; 424/729; 424/766; 424/736; 514/18; 514/458; 514/27; 514/456; 514/533

(57) ABSTRACT

Topical skin compositions include a complex containing components to provide a defense against the various pathway mechanisms of free radicals, reactive oxygen species, reactive nitrogen species, and other oxidizing species on the human body including the skin. The compositions may be administered by topically applying them in an amount to inhibit those mechanisms. The compositions and methods are directed to the prevention of the adverse or detrimental effects of free radicals, reactive oxygen species, reactive nitrogen species, and other oxidizing species on the human body including the skin. Thus, the compositions according to the invention improve barrier function, inhibit elastase and collagenase, and/or promote synthesis of collagen and elastin.

### CROSS REFERENCE TO RELATED APPLICATIONS

This application is a continuation-in-part application of U.S. application Ser. No. 10/155,305, filed May 24, 2002, which, in turn, is a continuation-in-part application of PCT/US00/31933. The entire contents of each of those applications is incorporated herein as if set forth fully again.

### BACKGROUND OF THE INVENTIONS

With an aging population, there has been an increase in the study of aging as it relates to the human body and, more particularly, human skin. For example, the treatment of aging skin exhibited by the presence of fine lines, wrinkles, and the like has received a great deal of attention. The dermal signs of aging such as fine lines, wrinkles, laxity, and hyperpigmentation have been fought through many tactics including surgery, laser treatment and cosmetics. Cosmetic treatments include the use of various creams and lotions to alter the effects of dermal aging. Much of the literature in the prior art focuses on the use of a single primary component to prevent one of several deleterious aging affects. For example, one tactic has been to use one or more hydroxy acids or retinoic acid to stimulate the re-growth of dermal cells, without other components. This approach is flawed because it does not recognize that aging is caused by the deleterious interaction of multiple agents on the skin, from multiple sources, causing damage to the skin through multiple simultaneous damage pathways.

More comprehensive studies have found that environmental factors, such as stress, sun exposure, and impurities in food, water, and air, also adversely effect components of the epidermal and dermal layers of the skin which, in turn, impact and alter the appearance of the skin and lead to an appearance of premature aging. For example, factors such as free radicals, reactive nitrogen species (“RNS”), reactive oxygen species (“ROS”), and other oxidizing species (“OOS”) that may or may not possess characteristics of each free radicals, RNS, and ROS, can adversely impact the human body including the skin. Particular factors within the groups noted above that have been found to impact and adversely affect the appearance of the skin include nitric oxide, superoxide radicals, hydrogen peroxide, and hydroxide free radicals. These factors have been variously implicated in a number of skin conditions including photodamage, general aging of the skin, contact dermatitis, wrinkling, lipid peroxidation, enzyme degradation, reduction and breakdown of collagen and/or elastin, degradation and inhibited reproduction of DNA, inflammation, and general damage to the skin tissue.

The ROS species include superoxide (O2—), hydrogen peroxide (H2O2), peroxy radicals (HO2 and RO2) alkyl peroxide (R2O2), hydroxyl radical (OH), alkoxy radical (OR), and singlet oxygen. The OOS species include hypohalous acids (HOX) (where X is chloride, bromide, iodide), Z-amines (where Z is either chlorinated or ammoniated amine containing compounds, the reactive nitrogen species (“RNS”) nitric oxide (NO), ammonia, cyclooxygenase, phospholipase A2, phospholipase C and transition metals.

Each of the ROS directly or acting as an intermediate are thought to act on cell membrane and/or other cellular components including organelles and their contents to adversely impact the skin. Thus, there is a need for a topical skin treatment composition and method that provides a defense against each of the ROS, RNS, and OOS noted above. In addition, it would be desirable if such a composition repaired damage caused by the ROS, RNS, and OOS noted above.

### SUMMARY OF THE INVENTIONS

The present inventions are directed to compositions that include selected components that provide a defense against the various pathway mechanisms of free radicals, reactive oxygen species, reactive nitrogen species, and other oxidizing species noted above that adversely effect the human body, including the skin. The inventions, therefore, also include methods for applying the compositions of the invention to the skin, to inhibit the causative factors that adversely effect the skin, and thereby treat and improve the quality of the skin. Generally, the compositions and methods of this invention are directed to the prevention of the adverse or detrimental effects of free radicals, reactive oxygen species, reactive nitrogen species, and other oxidizing species noted above, on the human body, including the skin. Thus, the present invention includes various compositions that include at least one anti-free radical component and/or an anti-superoxide component and/or an anti-hydrogen peroxide component and/or an anti-hydroxyl radical component and/or a chain breaking component.

Embodiments of the present invention include compositions that include a component that aids in cellular energy product and/or a component that aids in collagen synthesis and/or elastin synthesis and/or inhibits their degradation, and/or a component that aids in or provides cellular activity. For example, a composition of the present invention that has been found to positively effect one or more of the foregoing factors, includes a citrus component, such as a grapefruit component, such as grapefruit extract, a superoxide dismutase component, a glutathione component, a tetrahydrodiferuloylmethane component and/or a turmeric component, such as a tumeric extract, a bioflavonoid component, such as a citrus bioflavonoid component, a grape component, such as grape seed extract, a green tea component, such as a green tea extract, tocopherol, and/or a tocopheryl derivative such as tocopheryl acetate.

Another embodiment of the present invention that has similarly been found to positively effect factors that improve the health of skin, is a composition that includes a soybean component, such as a soybean protein component, a rice component, such as rice protein and more particularly hydrolyzed rice protein, and a sunflower seed component, such as a sunflower seed extract.

A further composition of the present invention includes a centella asiatica component, such as a centella asiatica extract, a corn kernel component, such as a corn kernel extract, a seaweed component, such as a seaweed extract, and ubiquinone (coenzyme Q).

Another composition of the present invention includes a rosemary component, such as a rosemary extract, a lecithin component, a ceramide component, such as a ceramide 3 component, a sitosterol component, such as beta sitosterol, a glycerin component, a panthenol component, a proline component, such as L-proline, and a hyaluronate component, such as sodium hyaluronate.

As explained further below, the present invention further includes compositions containing a combination of one or more of each of the foregoing composition components mentioned in the paragraphs above and, more particularly, the active agents contained therein.

Accordingly, methods of applying the compositions to the skin, to maintain and/or improve the condition of the skin of an individual for any of the reasons noted above, are also provided. Thus, the compositions may be applied to the skin for example, by topically applying an amount, such as an effective amount, of one or more of the various compositions according to the invention.

Processes for preparing the compositions according to the present invention are provided herein.

### BRIEF DESCRIPTION OF THE DRAWINGS

FIG. 1 is a graph showing the increase in erythema 30 minutes after UV exposure on human skin to which formulations were applied.

FIG. 2 is a graph showing the increase in erythema 10 hours after UV exposure on human skin to which formulations were applied.

FIG. 3 is a graph showing the effect of samples on procollagen secretion. The data are expressed as the collagen/viability ratio calculated by dividing the amount of procollagen detected in the tissue culture supernatants by WST-1 reduction as an indicator of cellular viability following the exposure period.

FIG. 4 is a graph showing the effect of samples on elastin secretion. The data are expressed as the elastin/viability ratio calculated by dividing the amount of elastin detected in the tissue culture supernatants by WST-1 reduction as an indicator of cellular viability following the exposure period.

FIG. 5 is a graph showing the effect of samples on MMP-1 activity. The data are expressed as % control MMP-1 activity. The horizontal line denotes 100% activity.

FIG. 6 is a graph showing the effect of samples on MMP-9 activity. The data are expressed as % control MMP-9 activity. The horizontal line denotes 100% activity.

FIG. 7 is a graph showing the effect of samples on Elastase activity. The data are expressed as % control elastase activity. The horizontal line denotes 100% activity.

FIG. 8 is a graph showing the effect of samples on NO production by RAW 264.7 cells. Data are expressed as % NO produced compared to the LPS stimulated positive control. L6=Lipochroman-6, NT=Nutriene tocotrienols, TQS=γ-Tocopherylquinone S, VC=Viapure Citrus, SZ=Soybean Zymbiosome, and NPR=NAB Pikea robusta.

FIG. 9 is a graph showing the effect of samples on lipid staining in HEK001 keratinocytes. Data are expressed as % control lipid from untreated cells.

### DETAILED DESCRIPTION OF THE INVENTIONS

As explained in the summary above, the present invention provides compositions that provide a defense mechanism against a variety of free radicals, reactive oxygen species, reactive nitrogen species, and other oxidizing species on the human body including the skin. These compositions assist in the maintaining and/or improving of the condition of the skin by, for example, increasing energy in cells of the skin and/or inhibiting adverse enzymes and/or maintaining and/or improving the quality and quantity of elastin, collagen, and glycosaminoglycan in the skin.

Compositions of the present invention will, generally speaking, include one or more of:

a citrus component, such as a grapefruit component, such as a grapefruit extract component, preferably a grapefruit peel extract, and preferably the component of apigenin;

a superoxide dismutase component;

a glutathione component;

a tetrahydrodiferuloylmethane component;

a phenolic component, such as a polyphenol component;

an essential oil component;

an ascorbic acid component;

a turmeric component, such as a tumeric extract;

a flavonoid component, such as a bioflavonoid component, such as a citrus bioflavonoid;

a grape component, such as grape seed extract;

a green tea component, such as a green tea extract;

tocopherol and/or derivatives thereof, such as tocopheryl acetate;

a soybean component, such as a soybean protein component;

a rice component, such as rice protein, and more particularly hydrolyzed rice protein;

a sunflower seed component, such as sunflower seed extract;

an octinoxate component,

a butyl methoxydibenzoyl-methane component;

a centella asiatica component, such as centella asiatica extract;

a corn kernel component, such as a corn kernel extract;

a seaweed component, such as a seaweed extract preferably laminaria digitata extract;

an ascorbyl tetraisopalmitate component;

a coenzyme component, such as ubiquinone (coenzyme Q);

a rosemary component, such as a rosemary extract, and preferably the component of ursolic acid;

a lecithin component;

a ceramide component, such as ceramide 3;

a beta sitosterol component;

a glycerin component;

a panthenol component, such as d-panthenol;

an adenosine component;

a proline component, such as L-proline;

a hyaluronate component, such as sodium hyaluronate;

a carbohydrate component;

a B vitamin component; and

a phylate component.

As used herein, the term “complex” means an admixture of various ingredients selected to focus around a common theme relating to the health and maintenance of mammalian skin. One such complex of ingredients could be focused on mediating effects of reactive oxygen and nitrogen species.

One particular embodiment of such a composition that is generally useful for its antioxidant property of preventing free radical damage to the skin, thereby protecting against the aging effects from free radical damage, includes the combination of a citrus component, and preferably a citrus component that contains apigenin. The citrus component may be derived from lemon, orange, tangerine, grapefruit, peppers, buckwheat, black currents, apricots, cherries, grapes and prunes. A preferred citrus component is a grapefruit component, and more particularly, a grapefruit extract that includes apigenin or simply is apigenin. It has been found that citrus components, and in particular citrus components that contain apigenin, such as a fruit extract and, more particularly, a grape fruit extract, inhibit damage caused by the reactive nitrogen species, in particular, nitric oxide (NO) production. The citrus component has been further found to inhibit lipid peroxidation, as well as inflammation caused by free radicals. Thus, compositions of the present invention that contain a citrus component, and in particular a grape fruit extract component, preferably containing apigenin, have been found to inhibit damage to the skin caused by nitric oxide production and/or lipid peroxidation and/or inflammatory factors such as inflammation caused by free radicals.

The composition generally further includes a superoxide dismutase component, which inhibits damage to proteins, elastin, collagen, and DNA, caused by superoxides that attack for example, enzymes; and a glutathione component, which inhibits damage caused by hydrogen peroxide. Additional components of this composition may include a phenolic component and/or one or more of the so-called “essential oils” and/or ascorbic acid (“vitamin C”) and/or tetrahydrodiferuloylmethane, which may, for example, be found in a tumeric component, such as a tumeric extract. Further components may include a flavonoid component, such as a bioflavonoid component, such as a citrus bioflavonoid component from, for example, grapefruit, lemon, or orange; and a polyphenol component which may, for example, be found in a grape component, such as grape seed extract, and preferably procyanidolic oligomers, a green tea component, preferably including polyphenols, and particularly epigallocatechin gallate (EGCG), tocopherol, and/or tocopheryl acetate, are each components that inhibit damage caused by hydroxyl radicals which attack lipids.

Thus, in one particular embodiment, this composition includes grapefruit extract in an amount of from about 0.01% to about 1%, superoxide dismutase in an amount of from about 0.0001% to about 0.01%, glutathione in an amount of from about 0.01% to about 1%, tetrahydrodiferuloy methane or a tumeric extract in an amount of from about 0.001% to about 1%, citrus bioflavonoids in an amount of from about 0.001% to about 1%, grape seed extract in an amount of from about 0.001% to about 1%, green tea extract in an amount of from about 0.01% to about 1%, tocopherol in an amount of from about 0.01% to about 2%, tocopheryl acetate in an amount of from about 0.01% to about 5%.

Thus, as will be appreciated, a composition generally as described above, may maintain and/or improve skin quality, thereby maintaining a youthful appearance, by reducing the detrimental effects of one or more of inflammation, lipid peroxidation, and degradation of collagen, elastin, and DNA.

In another particular embodiment of the present invention, a composition will generally include a soybean component, such as a soybean protein component, and preferably the isoflavones, such as genistein and daidzein. The soybean component has been found to be an inhibitor of the enzyme elastase, which is released to the skin in response to such factors as exposure the UV rays, dryness, and environmental stresses generally. Thus, the soybean component helps maintain and/or increase firmness and elasticity of the skin, particularly those that derive from the UV rays of sun exposure. This embodiment of the composition will generally also include a rice component, such as rice protein, and more particularly a hydrolyzed rice protein, which has an inhibitory effect on the enzyme collagenase. The inhibition of collagenase aids in protecting collagen in the skin, thereby maintaining and/or improving the condition of the skin with respect to elasticity, firmness, wrinkling, dryness, and age spots. A sunflower seed component, such as sunflower seed extract, may also be included in this embodiment. The sunflower seed component has been found to act as an anti-glycation factor, and to maintain and/or improve the condition of the skin by delaying the changes that cause collagen to become rigid with age and other detrimental factors discussed above. The composition may further include an octinoxate component and/or a butyl methoxydibenzyoylmethane component.

Thus, in one particular embodiment, this composition includes soybean protein (Glycine Soja) in an amount of from about 0.01% to about 3%, hydrolyzed rice protein in an amount of from about 0.01% to about 3%, sunflower seed extract in an amount of from about 0.01% to about 3%.

A further particular embodiment of the present invention, is a composition that includes a centella asiatica component, such as centella asiatica extract. The centella asiatica component has been found to promote collagen and elastin synthesis, thereby maintaining or improving the firmness, elasticity, and general strength of the skin. The primary active constituents are saponins (triterpenoids), that include asiaticoside, madecassoside, and madasiatic acid. A corn kernel component, such as a corn kernel extract, and more particularly myo-inositol, may be included in this embodiment, and it provides several benefits that include assistance in production and storage of energy in the cell, inhibition of lipid peroxidation, and it is generally a powerful antioxidant. Components of corn kernel extract that may separately or in combination be included in a composition, include nitrogenous elements, carbohydrates, B vitamins, trace elements, and/or myo-inositol in the form of phylate. A seaweed component, such as a seaweed extract (e.g., laminaria digitata extract), may be further included in this embodiment and, when included, it assists in increasing intercellular ATP rate and increasing oxygenation of cells and tissues, thereby generally increasing the structure of skin. Finally, a ubiquinone (coenzyme Q) component may be included and it acts as a coenzyme for various important enzymatic pathways particularly in the production of energy in cells, and optionally with ascorbyl tetraisopalmitate.

Thus, in one particular embodiment, this composition includes Centella Asiatica extract in an amount of from about 0.01% to about 3%, corn kernel extract in an amount of from about 0.01% to about 3%, seaweed extract in an amount of from about 0.01% to about 3%, coenzyme Q-10 in an amount of from about 0.001% to about 1%.

A further particular embodiment of the present invention is a composition that generally provides a hydrolipid matrix to the skin. This composition will generally include a rosemary component, such as a rosemary extract, and preferably rosmarinic acid, phenolic diterpenes, carnosol, carnosic acid, and/or ursolic acid, or simply is ursolic acid. The rosemary extract will preferably be an extract obtained form the leaf of a rosemary. The rosemary component will preferably be encapsulated in a liposome to enhance delivery. Additional components will generally include one or more of a lecithin component, a ceramide 3 component, a phospholipid such as a beta sitosterol component, a glycerin component, a panthenol component, a proline component, such as L-proline, and a hyaluronate component, such as sodium hyaluronate. Subcombinations of components of the above composition will preferably include a rosemary component, such as a rosemary extract, and preferably an extract of rosemary leaf, and a lecithin component. This subcombination aids in lipid retention and in forming a moisture layer barrier in and on the skin. A further subcombination of the above composition preferably includes a lecithin component, a ceramide 3 component, and a beta sitosterol component, or preferably includes a ceramide 3 component and a beta sitosterol component. This subcombination of the above components also aids in lipid retention and in forming a moisture layer barrier in and on the skin.

Thus, in one particular embodiment, this composition includes rosemary extract in an amount of from about 0.0001% to about 1%, a lipid complex that includes ceramide 3 in an amount of from about 0.001% to about 0.1% and a beta-sistosterol in an amount of from about 0.0001% to about 0.1%, glycerin in an amount of from about 0.1% to about 10%, panthenol in an amount of from about 0.01% to about 1%, proline in an amount of from about 0.001% to about 1%, and sodium hyaluronate in an amount of from about 0.001% to about 5%.

As will be appreciated, each of the foregoing compositions and subcombinations may be used alone, or may be used in combination with additional components to form a further new formulation. The present invention thus further includes compositions containing a combination of one or more of each of the foregoing composition components in further combination with additional components discussed below.

The compositions of the present invention may also include a cosmetically or pharmaceutically acceptable carrier. Components of the compositions may be encapsulated, such as in liposomal capsules. When a carrier is present, the complex forms from about 0.01% to about 10% by weight of the total composition, preferably from about 1% to about 7% by weight of the total composition.

In general, the anti-superoxide component may include those materials having anti-superoxide activity and, in particular, those having superoxide dismutase activity. In other words, it includes those components that can catalyze a dismutation reaction. For example, it includes superoxide dismutase (SOD), SODs modified by grafting polyalkylene oxide, polyethylene glycol, polysaccharide or acylated groups, salts of SOD, substances containing such SOD products, porphorins and materials with superoxide dismutase-like activity. In this respect, it includes those products mentioned in EP 223 257, the relevant contents of which are incorporated herein by reference.

All the superoxide dismustases described above, as well as the variants and equivalents that a person of skill in the art can deduce from the literature may be suitable as SODs for use in the present invention. In addition, they can be of differing origins. For example, they may be animal (bovine, porcine, and the like), human (blood), or plant (fungi, algae, spinach, and the like). They may also be obtained from bacteria or yeast, or alternatively by a biotechnological route. Examples of SODs that may have application in the present invention are described in U.S. Pat. No. 5,526,507, the contents of which is incorporated herein by reference. The SOD may form from about 0.0001% to about 5%, 0.01% to about 5% by weight of the complex. More, preferably, the SOD may be included in the complex in an amount from about 0.1% to about 2% by weight.

In general, the anti-hydrogen peroxide component may be a thiol or thiol derivative. In the context of the present invention, the term thiol is to be understood to be an organic compound characterized by the—SH group. Thiol derivatives are organic compounds that are either derivatives that retain the—SH group or are thio ethers or thio esters, in which case the—SH group is converted into the—SR group.

Compounds that are to be understood as being identical to the thiols or thiol derivatives according to the invention are those that are formed by tautomerism, di- or oligomerization by hydrogen bonding, hydration or other spontaneous rearrangement from the thiols or thiol derivatives. If a derivative is in equilibrium with an isomer by a different type of rearrangement, for example, migration of an alkyl group, this isomer is regarded as being included in the thiols and thiol derivatives of the invention.

Suitable thiol and thiol derivatives may include captopril, cysteamine, ergothioneine, mercaptopropionylglycine, penicillamine, N-acetylcysteine, S-acetylcysteine, N,S-diacetylcysteine, N,S-diacetylcysteinamide, cysteine ethyl ester, N-acetylcrysteine ethyl ester, S-acetylcysteine ethyl ester, N,S-diacetylcysteine ethyl ester, thioglycolic acid, cysteine, homocysteine, glutathione, thioglycerol, thiomalic acid, 2-mercaptopropionic acid, 3-mercaptopropionic acid, thiodiglycol, 2-mercaptoethanol, dithioreitol, thioxanthene, thiosalicylic acid, thiolactic acid, thiopropionic acid, thiodiglycolic acid, lipoic acid, and cosmetically acceptable salts thereof.

As used herein, the cosmetically acceptable salts include, but are not limited to alkali metal salts, e.g., sodium, lithium, potassium, and rubidium salts; alkaline earth metal salts, e.g., magnesium, calcium, and strontium salts; non-toxic heavy metal salts, e.g., aluminum and zinc salts; boron salts; silicon salts; ammonium salts; trialkylammonium salts, e.g., trimethylammonium and triethylammonium, and tetraalkylonium salts.

Generally, the anti-hydrogen peroxide component may be incorporated into the complex in an amount from about 0.001% to about 5% by weight, preferably from about 0.01% to about 2.5%, more preferably from about 0.1% to about 1% by weight of the complex.

Generally, anti-hydroxyl radical components can include one or more of the following: tocopherol, tocopherol derivatives, tetrahydrodiferuloylmethane, grape seed extract (e.g., vitis vinifera (grape) seed extract), grape fruit extract (e.g., citrus grandis (grapefruit) fruit extract), green tea extract (e.g., camellia sinensis (leaf) extract), turmeric acid, curcuminoids, tetrahydrocurcuminoids catechins, epigallocatechin 3-0-gallate and polyphenols, oligomeric proanthocyanidins, bioflavonoids, flavonoids, and mixtures thereof.

Tocopherol (Vitamin E) and its derivatives such as esters of tocopherol are useful in the composition of the present invention. Suitable tocopherols include the monomethyl, dimethyl, or triethyl derivatives of tocol, including but not limited to, alpha tocopherol, beta tocopherol, gamma tocopherol, delta tocopherol, epsilon tocopherol, zeta tocopherol, and eta tocopherol. Suitable esters of tocopherol include but are not limited to tocopheryl acetate, tocopheryl succinate, tocopheryl benzoate, tocopheryl propionate, tocopheryl sorbate, tocopheryl oleate, tocopheryl orotate, tocopheryl linoleate, tocopheryl nicotinate, and the 2-ethyl-hexanoate ester.

When the tocopherol and/or its derivatives are included in the complex of the present invention, they are used at level from about 0.01% to about 98%, preferably from about 0.01% to about 5%, and from 0.01% to about 2%.

Tetrahydrodiferuloylmethane and/or turmeric extract may also be incorporated into the complex at levels from about 0.1% to about 20% by weight of the complex, preferably from about 1% to about 10% by weight.

As discussed above, grape seed extract and complexes of grape seed extract with phospholipids may also be beneficial for use in the present invention. The extracts from grape seed include a mixture polyphenols such as epicatechin, proanthocyanidins, and catechins. A suitable complex of grape seed extract and phospholipid is described in U.S. Pat. No. 4,963,527, the contents of which are incorporated herein by reference.

When incorporated into the complex, the grape seed extract or its complex with phospholipids is present in an amount from about 0.001% to about 5% by weight of the complex, preferably from about 0.01% to about 2.5% by weight. Green tea extract may be included in the same amounts as the grape seed extract.

Flavonoids and bioflavonoids may also be useful in the present invention. It has been reported in Bravo, Polyphenols: Chemistry, Dietary Sources, Metabolism, and Nutritional Significance, Nutrition Reviews, Vol. 56, No. 11, 317-33 (November, 1998), the contents of which are incorporated herein by reference, that flavonoids may be subdivided into 13 classes shown below:

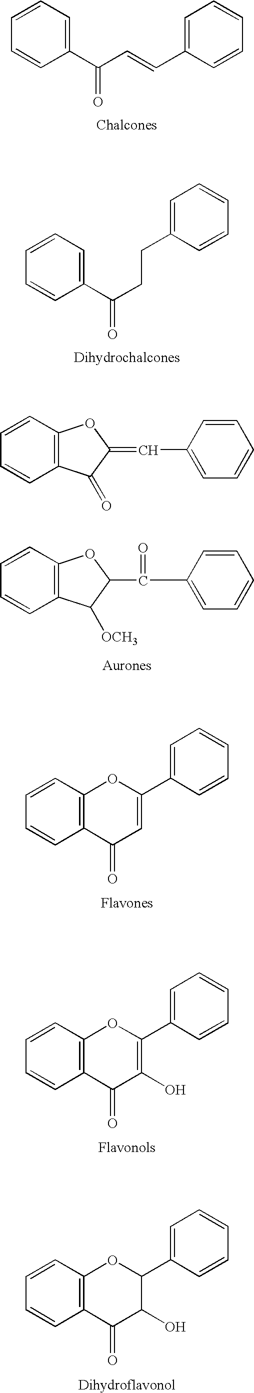

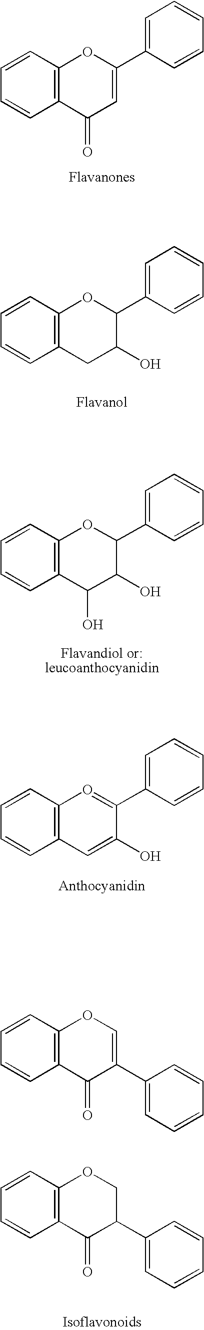

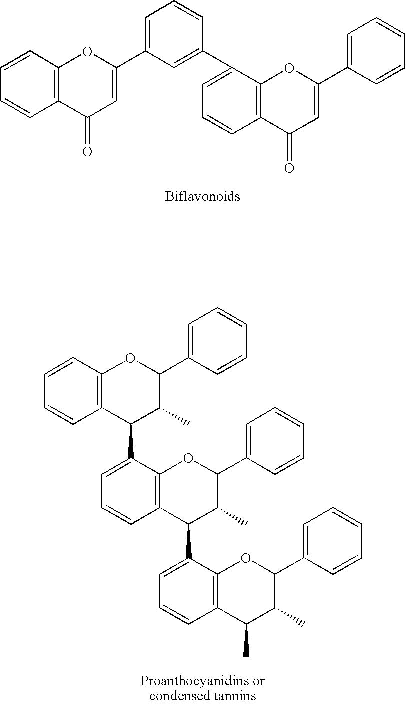

Flavonoids have, in general, the common structure of diphenylpropanes (C6-C3-C6) and consist of two aromatic rings linked through three carbons that usually form an oxygenated heterocycle. The basic structure is shown below:

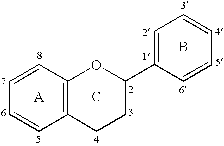

Flavonoids occasionally occur in plants as aglycones, although they are most commonly found as glycoside derivatives.

Specific suitable flavonoids for use in the present invention include but are not limited to rutin, citrin, quercitin, hesperidin, naringen, taxifolin, catechin, epicatechin, eriodictyol, naringenin, troxerutin, chrysin, tangeretin, luteolin, epigallocatechin, epigallocatechin gallate, fisetin, kaempferol, galangin, gallocetechin, epicatechin gallate, apigenin, diosmetin, myricetin, genistein, daidzein, or derivatives thereof. The flavonoids may be derived from any suitable source. A preferred source is from citrus.

When flavonoids are incorporated into the complex, they are present at a level from about 0.001% to about 20% by weight of the complex, preferably from about 0.01% to about 10% by weight.

Other specialty components may also be included such as palmitoyl hydroxypropyltrimonium amylopectin. In one embodiment, the palmitoyl hydroxypropyltrimonium amylopectin can be mixed with camellia sinensis extract. This may be present in amounts ranging from about 0.001% to about 2% by weight of the complex.

The chain breaker may include the same components as those described above for the anti-hydroxyl radical component. Thus, one or more of the above anti-hydroxyl radical components may also serve as a chain breaker component. Chain breaking antioxidants are those components that can break the chain reaction once lipid peroxidation is initiated.

As noted above, the complex composition may also include components selected to repair the damage caused by the ROS. In one embodiment, the compositions of the present invention includes at least one component that provides cellular energy production, at least one component that aids collagen synthesis, and/or at least one component that aids or provides cellular activity. These components may be used singly or, desirably, in combination.

A desirable cellular energy production component includes the ubiquinones. Ubiquinones are widely found in bacteria, fungi, yeasts, plants, and animals. It is known that different species produce isoforms (Q-n) with different numbers of isoprene units (n). For example, the number of isoprene units is 6 (Q6) in some microorganisms, nine (Q9) in plants, and ten (Q10) in humans. Coenzyme Q10 or 2,3,-dimethoxy-5-methyl-6-decaprenyl-benzoquinone functions to recover and maintain respiration and promotes ATP production in terms of energy supply for cellular activities. Derivatives of the ubiquinones such as ubiquinols may also be useful

The cellular energy production component, for example, coenzyme Q10, is incorporated into the complex in an amount ranging from about 0.001% to about 10%, preferably from about 0.01% to about 5% by weight of the complex.

To repair damage caused by ROS, it is desirable to include a component that will promote collagen synthesis. It has been suggested that hydroxy acids including alpha and beta hydoxy acids may be useful in this regard. As a result, the present invention contemplates including one or more alpha or beta hydroxy acids. Suitable examples include lactic, malic, glycolic, citric, and salicylic acid.

In addition, it has been found that ascorbic acid (Vitamin C) and its derivatives promote collagen synthesis. The ascorbic acid derivative useful in the present invention includes all enantiomers whether singly or in combination. Preferably, the ascorbic acid is provided in the levo form. In addition, the ascorbic acid or its derivatives may be in a water soluble or an oil soluble form.

Non-exclusive examples of the vitamin C (ascorbic acid) derivatives are, for instance, the alkyl esters of L-ascorbic acid where the alkyl portion has from 8 to 20 carbon atoms. With respect to the esters, they may be selected from the group consisting of fatty acid mono-, di-, tri- or tetra-esters of ascorbic acid. For example, such esters include, but are not limited to ascorbyl palmitate, ascorbyl laureate, ascorbyl myristate, ascorbyl stearate, ascorbyl dipalmitate, ascorbyl dilaurate, ascorbyl dimyristate, ascorbyl distearate, ascorbyl tripalmitate, ascorbyl trilaurate, ascorbyl trimyristate, ascorbyl tristearate, ascorbyl tetrapalmitate (tetrahexyldecyl ascorbate), ascorbyl tetralaurate, ascorbyl tetramyristate, ascorbyl tetrastearateL-ascorbyl palmitate, L-ascorbyl isopalmitate, L-ascorbyl dipalmitate, L-ascorbyl isostearate, L-ascorbyl distearate, L-ascorbyl diisostearate, L-ascorbyl myristate, L-ascorbyl isomyristate, L-ascorbyl 2-ethylhexanoate, L-ascorbyl di-2-ethylhexanoate, L-ascorbyl oleate and L-ascorbyl dioleate, tetrahexyl decyl ascorbate; phosphates of L-ascorbic acid such as L-ascorbyl-2-phosphate and L-ascorbyl-3-phosphate; sulfates of L-ascorbic acid such as L-ascorbyl-2-sulfate and L-acorbyl-3-sulfate; their salts with alkaline earth metals such as calcium and magnesium.

With respect to the salts, they may be selected from the phosphates and sulfates, preferably phosphate. The ascorbic acid phosphate is generally selected from L-ascorbic acid 3-phosphate, L-ascorbic acid 2-phosphate, L-ascorbic acid 3-pyrophosphate and bis (L-ascorbic acid 3,3-) phosphate. Preferably, the ascorbic acid phosphate is magnesium or sodium ascorbyl phosphate; more preferably, magnesium ascorbyl phosphate. Likewise, the ascorbic acid sulfate is generally selected from L-ascorbic acid 3-sulfate, L-ascorbic acid 2-sulfate, L-ascorbic acid 3-pyrosulfate and bis (L-ascorbic acid 3,3-) sulfate.

The collagen synthesis component, for example, the ascorbic acid and its derivatives, is incorporated in the complex in an amount ranging from about 0.001% to about 10%, preferably from about 0.01% to about 5% by weight of the complex.

It is believed that retinoids may affect cellular activity and thus it is desirable to incorporate retinoids in the complex of the present invention. The retinoids include retinol, retinal (Vitamin A aldehyde), and retinyl esters such as retinyl acetate, retinyl butyrate, retinyl propionate, retinyl octanoate, retinyl laurate, retinyl palmitate, retinyl oleate, and retinyl linoleate.

Retinoids tend to irritate the skin and therefore, it is desirable to incorporate them in the complex at levels so as to minimize the potential irritation. Alternatively, irritancy mitigants may be incorporated into the compositions to assist in preventing undue discomfort to the user while potentially permitting the dosage level of retinoid to be increased. Such irritancy mitigants include, but are not limited to ceramides, pseudoceramides, fatty acids, cholesterol, phospholipids, panthenol, oat extract, allantoin, ginkgo biloba, licorice extract, calendula, ginseng, butchers broom, and the like.

The cellular activity component, for example, the retinoid, is incorporated in the complex at a level ranging from about 0.001% to about 10%, preferably from about 0.01% to about 5% by weight of the complex.

The complex compositions according to the present invention are generally mixed with a pharmaceutically or cosmetically acceptable vehicle or carrier. The complex compositions of the present invention may be formulated as a solution, gel, lotion, cream, ointment, oil-in-water emulsion, water-in-oil emulsion, or other pharmaceutically or cosmetically acceptable form. The complex compositions of the present invention may also contain various known and conventional cosmetic components so long as they do not detrimentally affect the desired effects.

The pharmaceutically acceptable or cosmetically acceptable vehicle acts as a dilutant, dispersant, or carrier for other materials present in the complex composition, so as to facilitate their distribution when the complex composition is applied to the skin.

Vehicles other than water can include liquid or solid emollients, solvents, humectants, thickeners, and powders. For example, the following vehicles can be used alone or as a combination of one or more vehicles.

Vehicles may also include propellants such as propane, isobutane, dimethyl ether, carbon dioxide, nitrous oxide; and solvents such as ethyl alcohol, isopropanol, acetone, ethylene glycol monomethyl ether, diethylene glycol monobutyl ether, diethylene glycol monoethyl ether, or powders such as chalk, talc, fullers earth, kaolin, starch, gums, collodial silica, sodium polyacrylate, tetra alkyl and/or trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate.

Emollients, such as stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rapeseed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate.

As used herein, “emollients” refer to materials used for the prevention or relief of dryness, as well as for the protection of the skin. A wide variety of suitable emollients are known and may be used herein. Sagarin, Cosmetics, Science and Technology, 2^{nd} Edition, Vol. 1, pp. 3243 (1972), incorporated herein by reference, contains numerous examples of suitable materials.

The composition can optionally comprise sunscreens such as inorganic and organic sunscreens to provide protection from the harmful effects of excessive exposure to sunlight during use of the complex composition of the present invention. Examples of suitable sunscreens include those described in the U.S. OTC Sunscreen Monograph, such as octinoxate, and butyl methoxy dibenzoylmethane, the contents of which is incorporated herein by reference.

The composition of the invention can accordingly comprise from 0.1 to 10%, preferably from 1 to 5% by weight of an organic sunscreen material.

The composition optionally can also comprise as a sunscreen titanium dioxide or zinc oxide having an average particle size of from 1 to 300 nm, iron oxide having an average particle size of from 1 to 300 nm, silica, such as fumed silica having an average particle size of from 1 to 100 nm. It should be noted that silica, when used as an component in the emulsion according to the invention can provide protection from infrared radiation.

Ultrafine titanium dioxide in either of two forms, namely water-dispersible titanium dioxide and oil-dispersible titanium dioxide may be used. Water-dispersible titanium dioxide is ultrafine titanium dioxide, the particles of which are uncoated or which are coated with a material to impart a hydrophilic surface property to the particles. Examples of such materials include aluminum oxide and aluminum silicate. Oil-dispersible titanium dioxide is ultrafine titanium dioxide, the particles of which exhibit a hydrophobic surface property, and which, for this purpose, can be coated with metal soaps such as aluminum stearate, aluminum laurate, or zinc stearate, or with organosilicone compounds.

By “ultrafine titanium dioxide” is meant particles of titanium dioxide having an average particle size of less than 100 nm, preferably from 10 to 40 nm and most preferably from 15 to 25 nm. The total amount of titanium dioxide that can optionally be incorporated in the composition according to the invention is from 1 to 25%, preferably from 2 to 10% and ideally from 3 to 7% by weight of the composition.

A particularly convenient form of the composition is an emulsion, in which case an oil or oily material (emollient) will normally be present, together with an emulsifier to provide either a water-in-oil emulsion or an oil-in-water emulsion.

The composition can also comprise water, usually up to 95%, preferably from 5 to 95% by weight.

The composition can also optionally comprise a high molecular weight silicone surfactant that can also act as an emulsifier, in place of or in addition to the optional emulsifier(s) already mentioned.

The silicone surfactant may be a high molecular weight polymer of dimethyl polysiloxane with polyoxethylene and/or polyoxpropylene side chains having a molecular weight of from 10,000 to 50,000. When used, the dimethyl polysiloxane polymer is conveniently provided as a dispersion in a volatile siloxane, the dispersion comprising, for example, from 1 to 20% by volume of the polymer and from 80 to 99% by volume of the volatile siloxane. Ideally, the dispersion consists of a 10% by volume of the polymer dispersed in the volatile siloxane.

Examples of the volatile siloxanes in which the polysiloxane polymer can be dispersed include polydimethyl siloxane (pentamer and/or hexamer).

A preferred silicone surfactant is cyclomethicone and dimethicone copolyol, such as DC 3225C Formulation Aid available from DOW CORNING. Another is laurylmethicone copolyol, such as DC Q2-5200, also available from Dow Corning.

The amount of silicone surfactant, when present in the composition will normally be up to 25%, preferably from 0.5 to 15% by weight of the emulsion.

Examples of conventional adjuncts which can optionally be employed include preservatives, such as para-hydroxy benzoate esters; antioxidants, such butyl hydroxy toluene; humectants, such as glycerol, ethoxylated glycerins such as glycereth-26, sorbitol, 2-pyrrolidone-5-carboxylate, dibutylphthalate, gelatin, polyethylene glycol, such as PEG 200-600; buffers together with a base such as triethanolamine or sodium hydroxide; waxes, such as beeswax, ozokerite wax, paraffin wax; plant extracts, such as Aloe Vera, cornflower, witch hazel, elderflower, cucumber; as well as acerola cherry fermentate, thickeners; activity enhancers; colorants; and a fragrance, such as perfumes, may be included in a composition prepared in accordance with the present invention. Cosmetic adjuncts can form the balance of the composition.

It may also be desirable to incorporate anti-inflammatory and/or anti-irritant agents. The natural anti-inflammatory and/or anti-irritant agents are preferred. For example, licorice and its extracts, dipotassium glycyrrhizinate, oat and oat extracts, candelilla wax, alpha bisabolol, aloe vera, Manjistha (extracted from plants in the genus Rubia, particularly Rubia cordifolial), and Guggal (extracted from plants in the genus Commiphora, particularly Commiphora Mukul), may be used.

Skin conditioning agents that may be included, as mentioned above, are hyaluronic acid, its derivatives and salts including sodium hyaluronate, plant extracts such as kola nut, guarana mate, algae extract, proline, L-proline, and skin benefit agents such as ceramides, glycoceramides, pseudoceramides, sphingolipids such as sphingomyelins, cerebrosides, sulphatides, and ganglioside, sphingosines, dihydrosphingosine, phytosphingosines, phospholipids, either separately or in mixtures. Fatty acids may also be combined with these skin benefit agents. For example, the ceramides and glycoceramides include those described in U.S. Pat. Nos. 5,589,178, 5,661,118, and 5,688,752, the relevant portions of which are incorporated herein by reference. For example, the pseudoceramides include those described in U.S. Pat. Nos. 5,198,210, 5,206,020, and 5,415,855, the relevant disclosures of which are incorporated herein by reference.

Generally, compositions according to the present invention may be prepared in accordance with conventional procedures that are known in the art. For example, components of the present invention may be combined by sequential addition, with or without preference to order, followed by mixing to form a mixture. For example, components that are water soluble will generally be combined to form a water phase, and components that are fat soluble will generally be combined to form a fat phase. Thereafter, the two phases may be emulsified and then combined with carriers, etc. Alternatively, compositions may be prepared by admixing, such as in a one-pot system.

As noted above, the compositions of the present invention may be administered to an individual, preferably by topical application to the skin of the individual. The compositions may be applied in an amount effective to inhibit free radicals, reactive oxygen species, and other oxidizing species. Obviously, an individual may apply as much or as little of the composition as they desire or believe necessary but, for example, a composition of the present invention may be applied to the skin in an amount of about 1 mg/cm² to about 3 mg/cm² of skin. Preferably, the compositions of the present invention will be applied in an amount of about 2 mg/cm² per square inch of skin. Generally, the composition should be applied twice a day, such as in the morning and in the evening.

The compositions preferably include components for enhancing the transportation of the active components into the epidermal and dermal layers of the skin. Such components include dimethyl sulfoxide (DMSO) or n-decylmethyl sulfoxide (NDMS).

### EXAMPLES

The following examples are intended to illustrate, but not limit, the present invention. The examples below illustrate the effects of components of the ocmpositions of the present invention. They also set forth compositions according the present invention in combination with additional optional components that may alternatively be incorporated into any of the compositions set forth above.

### Example 1

Studies were performed to explore the effect of samples on collagen and elastin synthesis. Two assay systems were utilized for these studies. Human dermal fibroblasts, as these cells actively synthesize procollagen, and elastin.

Samples were diluted in media. RON SBD 101, Centella asiatica, and vitamin C were prepared at 0.001, 0.01, and 0.1% concentrations. The remaining samples were prepared at 0.1, 1, and 10% concentrations. Centella asiatica was prepared as an extract in DMSO:ethanol:water at 50:30:20. Human dermal fibroblasts (Hs-27) were plated in 24 well plates and were incubated overnight. The following day, the cells were treated with the samples at the concentrations previously indicated. Supernatant fluids were collected and tested for the presence of procollagen using a commercially available ELISA kit and elastin using the Fastin Elastin kit.

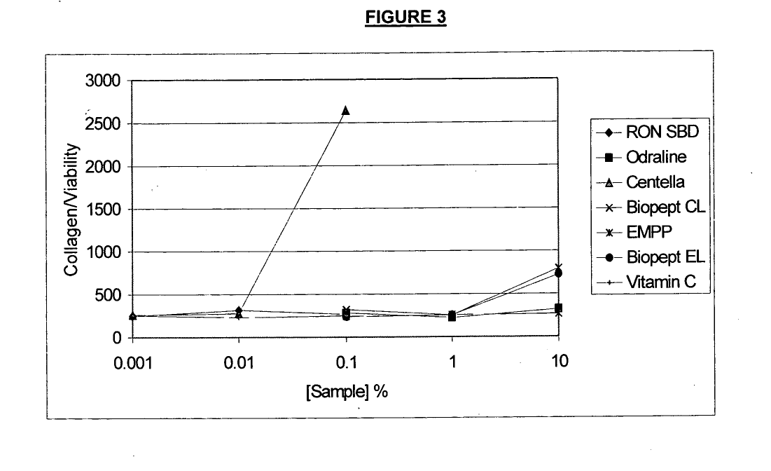

The levels of collagen produced by the cells are shown in FIG. 3. Collagen synthesis is expressed as a ratio of the amount of procollagen detected divided by viability to allow for any toxic effects of the samples. The data demonstrate that the Centella asiatica sample was most potent at inducing new collagen synthesis at a concentration of 0.1%. The Biopeptide CL and Biopeptide EL samples also induced a detectable increase in collagen synthesis at a concentration of 10%. The other samples had no detectable effect on procollagen synthesis.

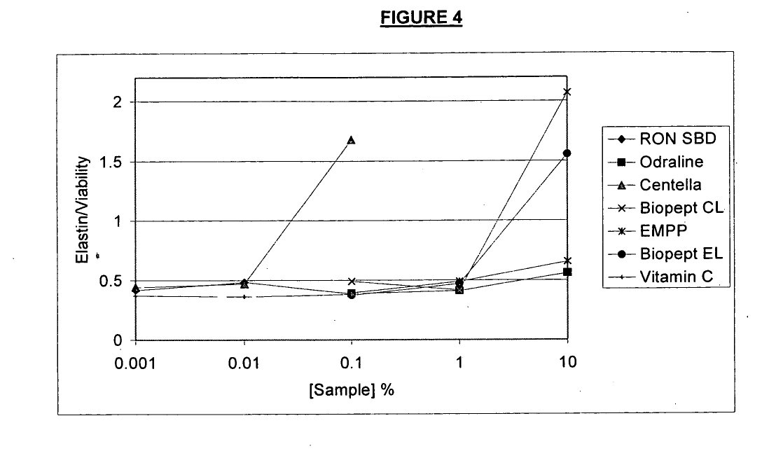

The data in FIG. 4 show the effect of the samples on elastin secretion. The data are again expressed as the ratio of the amount of elastin secreted divided by the viability of the cells at the time of supernatant collection. Like it did for collagen secretion, the Centella asiatica sample was the most potent inducer of elastin. Biopeptide CL and Biopeptide EL also induced detectable increases in elastin secretion. Finally, the Odraline and Biodynes EMPP samples induced slight increases in elastin at the highest concentration used (10%).

The results show that Centella asiatica is a potent inducer of both collagen and elastin. Additionally, the results suggest that the Biopeptide CL & EL samples induced both collagen and elastin although a high concentration of these materials is needed in order to induced the observed biological effect.

### Example 2

Sixteen samples were tested for their effects on three different enzymes (matrix metalloproteinases or MMPs) which are involved with breakdown of extracellular matrix proteins. Elhibin was the only sample that inhibited MMP-1. The most potent activators of MMP-1 were CoQ10, BVOSC Ascorbyl ester, Sophorine, Lemon bioflavonoids, ACTIMP 1.3.9, Lemon and mixed citrus extracts, and Kelpadelpie. Most of the samples had no effect of MMP-9, with only BVOSC ascorbyl ester being a strong inhibitor. The strongest inhibitors of elastase were Collalift, Alphinia leaf, Elhibin, Sophorine, Lemon bioflavonoids, ACTIMP 1.3.9, Lemon and mixed citrus extracts, Kelpadelpie, Extracellium, and Colhibin. Therefore, base on the desired profile of MMP-1 and elastase inhibition while having no effect of MMP-9, elhibin would be the raw material of choice.

The matrix metalloproteinases (MMP) are a group of zinc dependent enzymes, which degrade varying components of the extracellular matrix in both normal and diseased tissue. MMP-1 (interstitial collagenase) is thought to initiate the cleavage of fibrillar collagen while MMP-9 (gelatinase) digests the denatured collagen fragments generated by MMP-1. The products of MMP-9 digestion are then free to be incorporated into new collagen fibrils. Elastase breaks down elastin. The expression of these enzymes is under strict control and changes as individuals age or are exposed to UV irradiation. Because MMP-1 is involved in initiation of collagen breakdown, it would be advantageous for skin care products to inhibit MMP-1 activity. In contrast, such a skin care product should not inhibit MMP-9 as this would potentially inhibit synthesis of new collagen synthesis by blocking availability of collagen building blocks. Finally, elastase should be inhibited as to prevent digestion of elastin and the resulting elasticity of the skin.

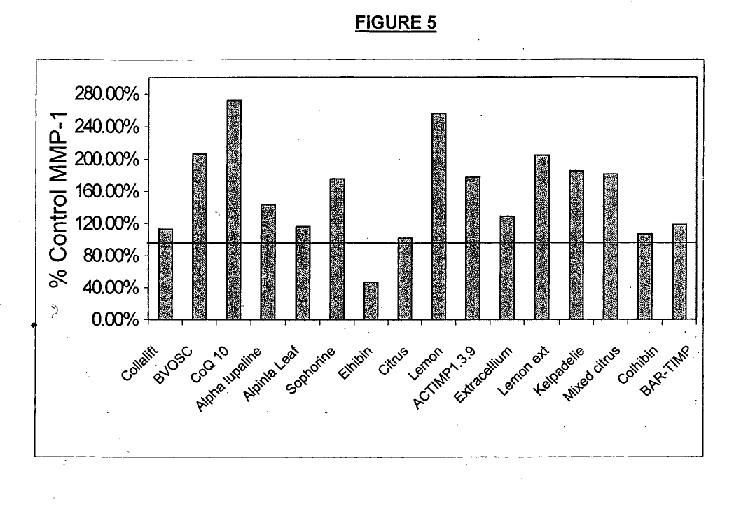

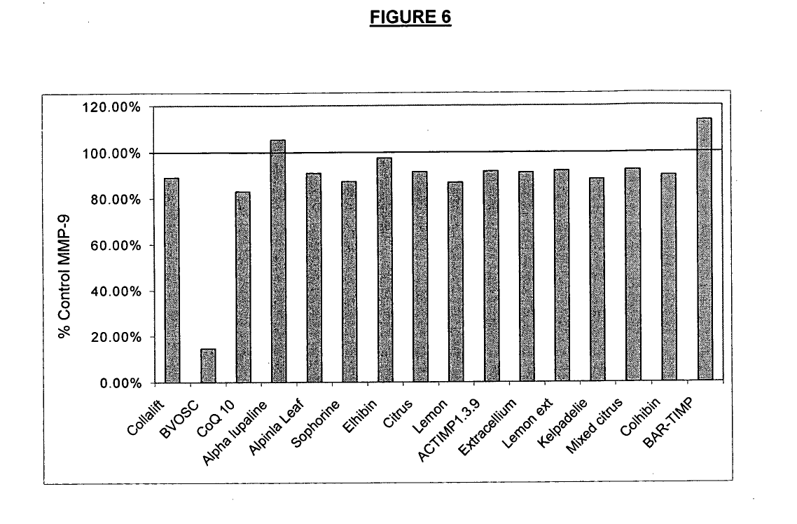

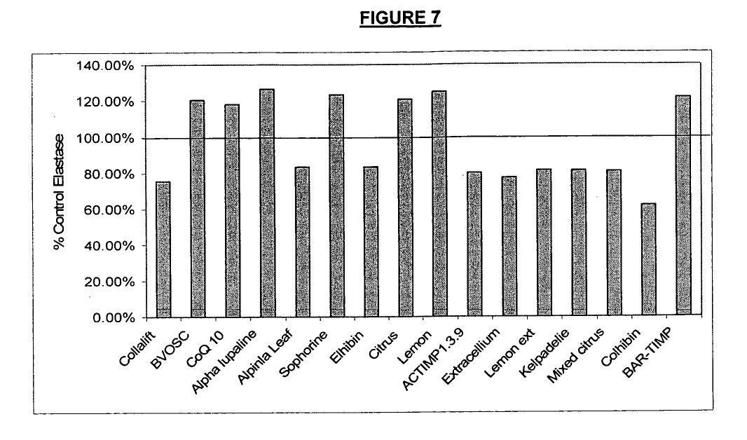

The data in Table I below gives information regarding the source and solubility for each of the samples tested. The data in FIG. 5 demonstrate the effect of the samples on MMP-1 activity. Elhibin was the only sample that inhibited MMP-1. The data in FIG. 6 demonstrate that most of the samples did not inhibit MMP-9. The only sample with strong inhibitory activity for MMP-9 was BVOSC ester. Finally, the data in FIG. 7 demonstrate that a number of the samples inhibited elastase. These samples were Collalift, Alphinia leaf, Elhibin, Sophorine, ACTIMP 1.3.9, Lemon and mixed citrus extracts, Kelpadelpie, Extracellium, and Colhibin.

TABLE I
Sample name, Supplier, Batch #, and solvent used for each sample.

| Sample | Supplier | Batch # | Solvent |
|---|---|---|---|
| Collalift Malt Extract | Coletica | 03020348 | PBS |
| BVOSC Ascorbylester | ABG | 11797RYA | DMSO/EtOH/ |
| | | | water |
| CoQ10 | ABG | 1190LM5A | DMSO |
| Alpha-Lupaline | Barnet | 982 | DMSO |
| Alpinia Leaf | Barnet | 010202 | PBS |
| Sophorine | Barnet | 5H636 | PBS |
| Elhibin | Pentapharm | 40197/301-02 | PBS |
| Citrus Bioflavonoids | ABG | 003-01 | PBS |
| Lemon Bioflavonoids | ABG | 1084X87A | PBS |
| ACTIMP 1.3.9 | Barnet | 105 | PBS |
| Extracellium | Coletica | 02120464 | PBS |
| Lemon Extract | Silab | 2-294-1 | PBS |
| Kelpadelie | Unknown | 104-182 | PBS |
| Mixed Citrus Extract | Silab | 2-179-2-1 | PBS |
| Colhibin | Pentapharm | 404652/325-01 | PBS |
| BAR-TIMP | Barnet | 030317 | PBS |

Commercially available kits were used for testing the effect of the samples on the activity of the MMPs of interest. For MMP-1, a kit from Amersham was used according to the manufacturer's specifications. For MMP-9 and Elastase, kits from Molecular Probes were used. The samples were prepared in the solvent noted in table I at stock concentrations of 100 mg/ml. The samples were diluted to 100 mg/ml using PBS.

### Example 3

Cells in skin can produce nitric oxide (NO) when exposed to UV light, and NO thus produced has the potential to induce age associated changes in skin. This study was performed in order to screen a panel of cosmetics and skin care raw materials for their effect on NO production by RAW 264.7 cells. The murine macrophage cell line RAW 264.7 was used in the study as it has been shown to produce NO when stimulated with LPS.

Murine RAW 264.7 cells were seeded in a 96 well plate at 1×10⁵ cells /well. The plate was incubated overnight. The following day, the cells were treated with the samples at 0.001, 0.01, and 0.1% for 2 hours. The samples are listed below in Table II. Following the exposure period, LPS was added to the wells at 100 ng/ml. The plate was incubated overnight. Equal volumes of culture supernatant and Griess reagent were incubated for 15 min at room temperature and the absorbance at 540 nm was read. The amount of nitrite in the samples was calculated from a standard curve generated with sodium nitrite.

TABLE II
Sample description.

| Sample | PD-ID | LIMS# | Solvent | Appearance |
|---|---|---|---|---|
| Lipochroman-6 | E23D92 | 200300444-1 | DMSO | Tannish, |
| | | | | crystalline |
| | | | | powder |
| Nutriene | 1999354700 | 200300444-2 | DMSO | Brown, clear, |
| Tocotrienols | | | | viscous liquid |
| g- | 00071319 | 200300444-3 | DMSO | Brown, clear, |
| Tocopheryl- | | | | viscous liquid |
| quinone S | | | | |
| Viapure Citrus | A90/02B001 | 200300444-4 | DMSO | Yellowish, fine |
| | | | | powder |
| Soybean | JQ1-124 | 200300444-5 | Water | Clear brown |
| Zymbiosome | | | | liquid |
| NAB Pikea | 46280 | 200300444-6 | Water | Clear, yellow |
| Robusta | | | | liquid |

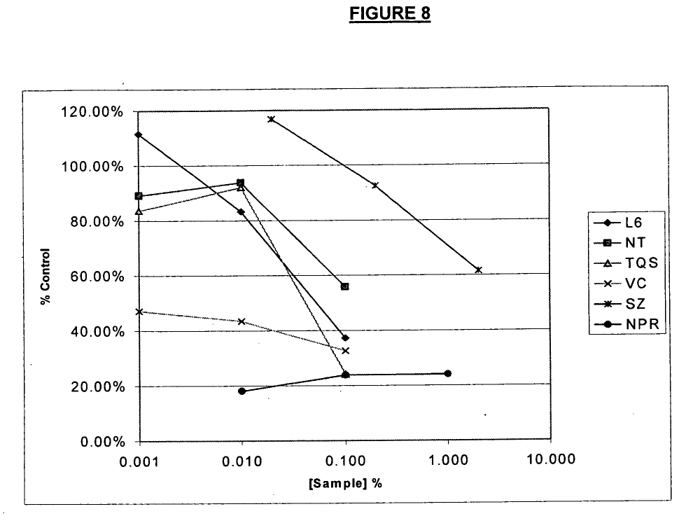

The data shown in Table III below and FIG. 8 show that all of the samples tested had inhibitory effects on nitrite accumulation in supernatants of LPS stimulated RAW cells. The samples all had inhibitory activity.

TABLE III
NO production by RAW 264.7 cells stimulated with LPS. Data are shown as
ng nitrite/ml supernatant.

| | | | | Nitrite produced |
|---|---|---|---|---|
| Sample | PD-ID | LIMS# | Doses | ng/ml |
| Negative control | NA | NA | NA | 0 |
| LPS control | NA | NA | 100 ng/ml | 901.7 |
| Lipochroman-6 (L6) | E23D92 | 200300444-1 | 0.001, 0.01, 0.1% | 1006.5, 750.7, 336.7 |
| Nutriene Tocotrienols | 1999354700 | 200300444-2 | 0.001, 0.01, 0.1% | 804.1, 845.7, 504 |
| (NT) | | | | |
| g-Tocopherylquinone | 00071319 | 200300444-3 | 0.001, 0.01, 0.1% | 754.2, 830.3, 217.9 |
| S (TQS) | | | | |
| Viapure Citrus (VC) | A90/02B001 | 200300444-4 | 0.001, 0.01, 0.1% | 425.4, 391.7, 294.9 |
| Soybean Zymbiosome | JQ1-124 | 200300444-5 | 0.02, 0.2, 2.0% | 1053.6, 834.1, 554.8 |
| (SZ) | | | | |
| NAB Pikea Robusta | 46280 | 200300444-6 | 0.01, 0.1, 1.0% | 164, 214.9, 216.9 |
| (NPR) | | | | |

The results show that all of the samples had an inhibitory effect on the accumulation of nitrite in the culture supernatants. The most potent samples were lipochroman-6, Vitapure citrus, and g-tocopherylquinone S. The aqueous samples Pikea robusta and Soybean Zymbiosome both exhibited inhibitory in this experiment as they were used at higher concentrations than those used in the previous experiment. It is suspected that the high inhibitory activity seen in Pikea treated cells was due to a dilution error. Finally, it appears that the tocotrienols (NT) and synthetic tocopherol (L6) have more inhibitory activity than do mixed tocopherols

### Example 4

Four new samples that boost cellular energy were tested for their ability to augment extracellular matrix component production in response to Centella and Biodynes. Human dermal fibroblasts were used as these cells actively synthesize extracellular matrix components. In addition to measuring procollagen and elastin levels, hyaluronic acid levels were also measured. Hyaluronic acid is a member of the glycosaminoglycan family of compounds. Glycosaminoglycans make up the ground substance of connective tissue, and along with elastin, help provide elasticity to skin. They also hold water and therefore provide viscosity and hydrating properties.

Samples were diluted in media. Centella asiatica and vitamin C were prepared at 0.001%. Biodynes EMPP was prepared at 0.1%. Centella asiatica was prepared as an extract in DMSO:ethanol:water at 50:30:20. The “energy booster” samples, Seanergilium algae extract, Thiotaine, Sepitonic, and Phytovityl corn kernel extract, were all prepared in media at 0.01, 0.1, and 1.0%. Human dermal fibroblasts (Hs-27) were plated in 24 well plates and were incubated overnight. The cells were treated with the samples at the concentrations indicated for 2 consecutive days. Supernatant fluids were collected and tested for the presence of procollagen and hyaluronic acid using commercially available ELISA kits (Takara and Corgenix respectively) and elastin using the Fastin Elastin kit (Biocolor).

The levels of procollagen produced by the cells are shown in Table IV. The data demonstrate that none of the energy booster samples had a positive effect on secretion of procollagen by the cells. In contrast, the energy booster samples had no effect on or actually inhibited procollagen secretion by unstimulated and stimulated cells. The only exception was cells treated with Seanergilium produced more procollagen than untreated negative control cells

TABLE IV
Effect of samples on procollagen secretion. Data are expressed
as ng/ml procollagen/ml supernatant calculated from a standard
curve generated with the procollagen standard provided with the
ELISA kit.

| | | Centella | Biodynes | |
|---|---|---|---|---|
| | Control | asiatica | EMPP | Vitamin C |
| Media | 1533 | 1759 | 1518 | 1744 |
| Seanergilium | 1696 | 1781 | 1428 | 1325 |
| Sepitonic | 1381 | 1347 | 1297 | 1309 |
| Thiotaine | 1367 | 1347 | 1528 | 1352 |
| Phytovityl | 1221 | 1196 | 965 | 1055 |

The data in Table V show the effect of the samples on elastin secretion. An increase in Biodynes EMPP stimulated elastin secretion was seen when Sepitonic and Thiotaine were the co-stimuli. Finally, the supernatants were analyzed for the presence of hyaluronic acid. The data in Table VI show the effect of the samples on hyaluronic acid secretion by the cells. The data demonstrate that Sepitonic and Phytovityl both augmented hyaluronic acid secretion by cells stimulated with Centella asiatica and Biodynes EMPP. In contrast, Seanergilium and Thiotaine both inhibited hyaluronic acid secretion.

TABLE V
Effect of samples on elastin secretion. Data are expressed
as the % media control elastin calculated by dividing the amount
of elastin in detected in the tissue culture supernatants from
treated cells by the amount of elastin secreted by untreated
control cells.

| | | Centella | Biodynes | |
|---|---|---|---|---|
| | Control | asiatica | EMPP | Vitamin C |
| Media | 100 | 115 | 107 | 148 |
| Seanergilium | 107 | 117 | 111 | 112 |
| Sepitonic | 119 | 110 | 118 | 116 |
| Thiotaine | 107 | 107 | 122 | 112 |
| Phytovityl | 138 | 100 | 110 | 94 |

The above Table IV below shows the effect of samples on hyaluronic acid secretion. Data are expressed as ng hyaluronic acid/ml culture supernatant. The amount of hyaluronic acid in each supernatant was calculated from a standard curve generated using a hyaluronic acid standard supplied with the ELISA kit.

| | | Centella | Biodynes | |
|---|---|---|---|---|
| | Control | asiatica | EMPP | Vitamin C |
| Media | 1112 | 989 | 862 | 633 |
| Seanergilium | 694 | 687 | 691 | 789 |
| Sepitonic | 1175 | 1296 | 1316 | 1226 |
| Thiotaine | 692 | 636 | 743 | 916 |
| Phytovityl | 1449 | 1205 | 1520 | 943 |

The most dramatic results in this experiment were the effect of some of the energy booster samples on hyaluronic acid secretion. Both Sepitonic and Phytovityl induced hyaluronic acid alone. Secretion was augmented by both Centella asiatica and Biodynes EMPP in the presence of Sepitonic and by Biodynes EMPP in the presence of Phytovityl. Sepitonic also enhanced biodynes stimulated elasin secretion but inhibited collagen secretion. Phytovityl on the other hand inhibited both collagen and elastin secretion. Seanergilium had little effect on secretion of any of the matrix components, and Thiotaine only enhanced Biodynes stimulated elastin secretion. In conclusion therefore, due to hyaluronic acids properties of providing hydration, viscosity, and elasticity, the Sepitonic or Phytovityl materials may be valuable for skin applications where an increase in hyaluronic acid, and subsequently increased hydration, is desired.

### Example 5

Keratinocytes treated with Urlisomes and Merospheres V overnight had higher intracellular lipid levels than untreated control cells. It appeared that Urlisomes induced a higher level of lipid incorporation than did Merospheres, but this could be due to a difference in lipid concentrations of the two products. There did not seem to be any non-specific staining as visual inspection of the cells following staining, but prior to stain extraction, showed multiple intracellular lipid droplets.

Dryness can be an irritating problem with skin, and it results from loss of water from the skin. The ability to retain water is associated with lipid content of the skin, especially in the stratum corneum. Thus it seems reasonable that if the lipid content in keratinocytes, the primary cell type found in the stratum corneum, could be raised, water loss might be prevented and thus alleviate dry skin. To test this possibility, two lipid-containing samples were tested for their ability to augment the lipid levels of cultured keratinocytes.

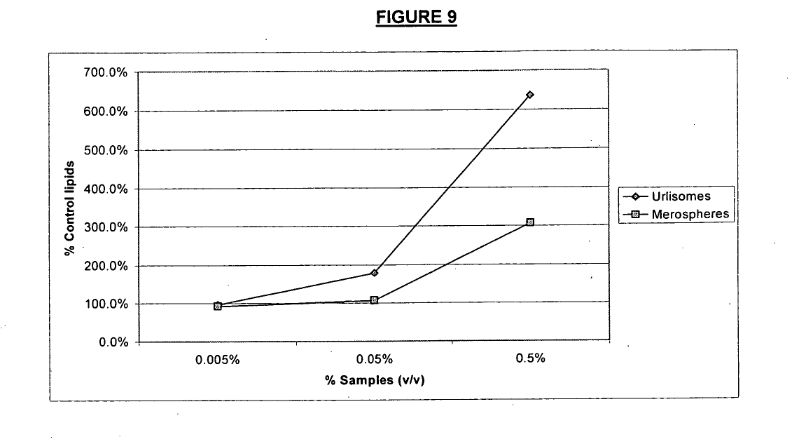

The data from a representative experiment shown in FIG. 9 demonstrate that exposure of the cells to both samples resulted in increased lipid staining. Urlisomes seemed to have a greater effect on lipid levels than did Merospheres V. However, this could simply be due to a difference in lipid content of the two samples. Alternatively, the difference could result from better uptake of the lipids in the Urlisome sample compared to the Merosphere V sample.

Human HEK001 cells were plated at 2×10⁴/well in 96 well plates and were incubated overnight. The following day, the cells were exposed to the samples that had been diluted into cell culture media at 0.005%, 0.05%, and 0.5%. The cells were then again incubated overnight. The following day, the cells were fixed in 1% formaldehyde. Cellular lipids were then stained with Oil Red O stain (1). Following staining, the lipid bound stain was extracted with isopropanol. The OD of the extracted stain was read at 515 nm.

### Example 6

The following is an example of a preferred composition according to the present invention.

Component

Octinoxate
Avobenzone
Glycerin
Panthenol
Proline
Sodium Hyaluronate
Glycerin (&) Lecithin (&) Ceramide 3 (&) Beta-Sitosterol
Water (&) Rosmarinus Officinalis (Rosemary) Leaf Extract (&)
Lecithin
Soybean (Glycine Soja) Protein
Hydrolyzed Rice Protein
Sunflower Seed Extract
Superoxide Dismutase
Glutathione
Tocopherol
Tocopherol Acetate
Tetrahydrodiferuloylmethane
Palmitoyl Hydroxypropyltrimonium Amylopectin/Glycerin
Crosspolymer (and) Lecithin (and) Camellia Sinensis Extract
Citrus Grandis (Grapefruit) Fruit Extract
Grape (Vitis Vinifera) Seed Extract (&) Phospholipids
D.I. Water
Glyceryl Polymethacrylate
Butylene Glycol
Potassium Cetyl Phosphate (&) Hydrogenated Palm
Glycerides
Arachidyl Alcohol (&) Behenyl Alcohol (&) Arachidyl Glucoside
PEG-8 Dimethicone
Hydroxyethylacrylate (&) Sodium Acryloyldimethyl Taurate
Copolymer (and) Squalane (&) Polysorbate 60
Phenoxyethanol (&) Methylparaben (&) Ethylparaben (&)
Propylparaben (&) Butylparaben (&) Isobutylparaben
Disodium EDTA
Phenoxyethanol (&) Iodopropynyl Butylcarbamate
Aloe Vera Gel
Bioflavonoids
C12-15 Alkyl Benzoate & Dipropylene Glycol Dibenzoate (&)
PPG-15 Stearyl Ether Benzoate
Dimethicone
Tetrahexyldecyl Ascorbate
Fragrance Camomille Day 451101

### Example 6

The following is example of another composition according to the present invention.

Component

Octinoxate
Avobenzone
Glycerin
Panthenol
Proline
Sodium Hyaluronate
Glycerin (&) Lecithin (&) Ceramide 3 (&) Beta-Sitosterol
Water (&) Rosmarinus Officinalis (Rosemary) Leaf Extract (&)
Lecithin
Soybean (Glycine Soja) Protein
Hydrolyzed Rice Protein
Sunflower Seed Extract
Superoxide Dismutase
Glutathione
Tocopherol
Tocopherol Acetate
Tetrahydrodiferuloylmethane
Palmitoyl Hydroxypropyltrimonium Amylopectin/Glycerin
Crosspolymer (and) Lecithin (and) Camellia Sinensis Extract
Citrus Grandis (Grapefruit) Fruit Extract
Grape (Vitis Vinifera) Seed Extract (&) Phospholipids
D.I. Water
Butylene Glycol
Glyceryl Stearate (&) PEG 100 Stearate
Arachidyl Alcohol (&) Behenyl Alcohol (&) Arachidyl Glucoside
Behenyl Alcohol
Cetyl Alcohol
Ozorerite
Hydroxyethylacrylate (&) Sodium Acryloyldimethyl Taurate
Copolymer (and) Squalane (&) Polysorbate 60
Methylparaben
Disodium EDTA
Benzyl Alcohol
Chlorphensin
Aloe Vera Gel
Bioflavonoids
Isostearyl Palmitate
Squalane
FinSun
Caprylic/capric triglycerides
Dimethicone
Stearyl Glycyzzininate
Tetrahexyldecyl Ascorbate
Fragrance Camomille Day 451101

### Example 7

The following is also composition that can be prepared according to a further embodiment of the present invention.

Component

Glycerin
Panthenol
Proline
Sodium Hyaluronate
Glycerin (&) Lecithin (&) Ceramide 3 (&) Beta-Sitosterol
Water (&) Rosmarinus Officinalis (Rosemary) Leaf Extract (&)
Lecithin
Centella Asiatica
Water (&) Zea Mays (Corn) Kernel Extract
Laminaria Digitata Extract (&) Butylene Glycol
Superoxide Dismutase
Glutathione
Tocopherol
Tocopherol Acetate
Tetrahydrodiferuloylmethane
Palmitoyl Hydroxypropyltrimonium Amylopectin/Glycerin
Crosspolymer (and) Lecithin (and) Camellia Sinensis Extract
Citrus Grandis (Grapefruit) Fruit Extract
Grape (Vitis Vinifera) Seed Extract (&) Phospholipids
D.I. Water
Glyceryl Polymethacrylate
Butylene Glycol
Potassium Cetyl Phosphate (&) Hydrogenated Palm
Glycerides
Arachidyl Alcohol (&) Behenyl Alcohol (&) Arachidyl Glucoside
PEG-8 Dimethicone
Hydroxyethylacrylate (&) Sodium Acryloyldimethyl Taurate
Copolymer (and) Squalane (&) Polysorbate 60
Phenoxyethanol (&) Methylparaben (&) Ethylparaben (&)
Propylparaben (&) Butylparaben (&) Isobutylparaben
Disodium EDTA
Phenoxyethanol (&) Iodopropynyl Butylcarbamate
Bioflavonoids
C12-C15 Alkyl Ethyl Hexanote
Ubiquinone
Tetrahexyldecyl Ascorbate
Fragrance Camomille Night 451100

### Example 8

The following is an example of a further composition that can be prepared according to the present invention.

Component

Glycerin
Panthenol
Proline
Sodium Hyaluronate
Glycerin (&) Lecithin (&) Ceramide 3 (&) Beta-Sitosterol
Water (&) Rosmarinus Officinalis (Rosemary) Leaf Extract (&)
Lecithin
Centella Asiatica
Water (&) Zea Mays (Corn) Kernel Extract
Laminaria Digitata Extract (&) Butylene Glycol
Superoxide Dismutase
Glutathione
Tocopherol
Tocopherol Acetate
Tetrahydrodiferuloylmethane
Palmitoyl Hydroxypropyltrimonium Amylopectin/Glycerin
Crosspolymer (and) Lecithin (and) Camellia Sinensis Extract
Citrus Grandis (Grapefruit) Fruit Extract
Grape (Vitis Vinifera) Seed Extract (&) Phospholipids
D.I. Water
Butylene Glycol
Glyceryl Stearate (&) PEG 100 Stearate
Arachidyl Alcohol (&) Behenyl Alcohol (&) Arachidyl Glucoside
Behenyl Alcohol
Cetyl Alcohol
Carbomer 980
Triethanolamine
Diazolidinyl Urea (and) Iodopropynyl Butylcarbamate
(Replaces R4161)
Phenoxyethanol
Aloe Vera Gel
Bioflavonoids
Isostearyl Palmitate
Squalane
C12-15 Alkyl Benzoate & Dipropylene Glycol Dibenzoate (&)
PPG-15 Stearyl Ether Benzoate
Caprylic/capric triglycerides
Dimethicone
Ubiquinone
Tetrahexyldecyl Ascorbate
Fragrance Camomille Night 451100

### Example 9

The following is a topical skin composition according to one embodiment of the present invention. Unless otherwise indicated, for each of the following examples, percentages are by weight.

| Component | Wt. % |
|---|---|
| D.I. Water | 56.595 |
| Anti-superoxide component (superoxide | 0.005 |
| dismutase) | |
| Anti-hydrogen peroxide component (glutathione) | 0.2 |
| Anti-hydroxyl radical component (tocopheryl | 1.0 |
| acetate) | |
| Anti-hydroxyl radical component (tocopherol) | 0.2 |
| Anti-hydroxyl radical component | 0.1 |
| (tetrahydrodiferuloylmethane) | |
| Anti-hydroxyl radical component (Grape (Vitis | 0.1 |
| Vinifera) Seed Extract (&) Phospholipids) | |
| Anti-hydroxyl radical component (Bioflavonoids) | 0.1 |
| Anti-hydroxyl radical component (Palmitoyl | 0.1 |
| Hydroxypropyltrimonium Amylopectin/Glycerin | |
| Crosspolymer (and) Lecithin (and) Camellia Sinensis Extract) | |
| Emollient(s) | 21.5 |
| Humectant(s) | 5.205 |
| Emulsifier(s) | 2.3 |
| Skin conditioning agent(s) | 0.1 |
| Sunscreen(s) (UVA) | 3.0 |
| Sunscreen(s) (UVB) | 7.5 |
| Thickener(s) | 0.3 |
| Ph modifier(s) | 0.3 |
| Preservative(s) | 1.25 |
| Fragrance(s) | 0.1500 |
| Total | 100.000 |

### Example 10

The following is a topical skin composition according to one embodiment of the present invention. In this embodiment, the composition provides a defense against ROS and also includes components to help repair damage caused ROS.

| Component | Wt. % |
|---|---|
| D.I. Water | 57.635 |
| Anti-superoxide component (superoxide dismutase) | 0.005 |
| Anti-hydrogen peroxide component (glutathione) | 0.2 |
| Anti-hydroxyl radical component (tocopheryl | 1.0 |
| acetate) | |
| Anti-hydroxyl radical component (tocopherol) | 0.2 |
| Anti-hydroxyl radical component | 0.1 |
| (tetrahydrodiferuloylmethane) | |
| Anti-hydroxyl radical component (Grape (Vitis | 0.1 |
| Vinifera) Seed Extract (&) Phospholipids) | |
| Anti-hydroxyl radical component (Bioflavonoids) | 0.1 |
| Anti-hydroxyl radical component (Palmitoyl | 0.1 |
| Hydroxypropyltrimonium Amylopectin/Glycerin Crosspolymer | |
| (and) Lecithin (and) Camellia Sinensis Extract) | |
| Cellular activity component (retinyl acetate) | 0.16 |
| Cellular energy production component (Ubiquinone) | 0.05 |
| Collagen synthesis component (tetrahexyldecyl | 0.1 |
| ascorbate) | |
| Emollients | 26.5 |
| Humectants | 5.3 |
| Emulsifiers | 2.3 |
| Skin conditioning agent(s) | 0.1 |
| Silica (12 micron) | 2.0 |
| Silica (3 micron) | 2.0 |
| Aloe vera gel | 1.0 |
| Thickener(s) | 0.3 |
| Ph modifier(s) | 0.3 |
| Preservative(s) | 0.3 |
| Fragrance | 0.150 |
| Total | 100.00 |

### Example 11

The following is a topical skin composition according to one embodiment of the present invention. In this embodiment, the composition provides a defense against ROS and also includes components to help repair damage caused ROS.

| Component | Wt. % |
|---|---|
| D.I. Water | 66.68 |
| Anti-superoxide component (superoxide dismutase) | 0.005 |
| Anti-hydrogen peroxide component (glutathione) | 0.2 |
| Anti-hydroxyl radical component (tocopheryl | 1.0 |
| acetate) | |
| Anti-hydroxyl radical component (tocopherol) | 0.2 |
| Anti-hydroxyl radical component | 0.1 |
| (tetrahydrodiferuloylmethane) | |
| Anti-hydroxyl radical component (Grape (Vitis | 0.1 |
| Vinifera) Seed Extract (&) Phospholipids) | |
| Anti-hydroxyl radical component (Bioflavonoids) | 0.1 |
| Anti-hydroxyl radical component (Palmitoyl | 0.1 |
| Hydroxypropyltrimonium Amylopectin/Glycerin Crosspolymer | |
| (and) Lecithin (and) Camellia Sinensis Extract) | |
| Cellular activity component (retinyl acetate) | 0.16 |
| Cellular energy production component (Ubiquinone) | 0.05 |
| Collagen synthesis component (tetrahexyldecyl | 0.1 |
| ascorbate) | |
| Emollients | 1 |
| Humectants | 1.65 |
| Emulsifiers | |
| Skin conditioning agent(s) | 0.1 |
| Thickener(s) | 0.2 |
| Ph modifier(s) | |
| Preservative(s) | 0.3 |
| Fragrance | 0.15 |
| Cyclomethicone | 10.00 |
| Polyglycerylmethacrylate | 10.00 |
| Dimethicone Copolyol | 2.00 |
| 12 micron Silica | 2.00 |
| 3 micron Silica | 2.00 |
| Polyacrylamide (and) C₁₃₋₁₄ Isoparaffin (and) | 1.00 |
| Laureth-7 | |
| Polysorbate 20 | 0.50 |
| Total | 100.00 |

### Example 12

The following tests were performed to determine the effect of providing a complex composition according to the present invention in comparison to a placebo, Vitamin E, and Vitamin C. The tests were conducted by outlining a number of two inch sections on the back of a human. The following formulas were applied in a randomized manner to the sections.

| | Wt. % | Wt. % | Wt. % | Wt. % |
|---|---|---|---|---|
| Component | A | B | C | D |
| Emollient(s) | 21.5 | 21.5 | 21.5 | 21.5 |
| Humectant(s) | 6.205 | 6.205 | 6.205 | 6.205 |
| Emulsifier(s) | 1.3 | 1.3 | 1.3 | 1.3 |
| Skin conditioning agent(s) | 0.1 | 0.1 | 0.1 | 0.1 |
| Thickener(s) | 0.3 | 0.3 | 0.3 | 0.3 |
| Ph Modifier(s) | 0.3 | 0.3 | 0.3 | 0.3 |
| Preservative(s) | 1.25 | 1.25 | 1.25 | 1.25 |
| Fragrance(s) | 0.15 | 0.15 | 0.15 | 0.15 |
| Tocopherol | | 1.2000 | | 0.2000 |
| Glutathione | | | | 0.2000 |
| Tetrahydrodiferuloylmethane | | | | 0.1000 |
| Grape (Vitis Vinifera) Seed | | | | 0.1000 |
| Extract (&) Phospholipids | | | | |
| Bioflavonoids | | | | 0.1000 |
| Palmitoyl | | | | 0.1000 |
| Hydroxypropyltrimonium | | | | |
| Amylopectin/Glycerin | | | | |
| Crosspolymer (and) Lecithin | | | | |
| (and) Camellia Sinensis Extract | | | | |
| Superoxide dismutase | | | | 0.0050 |
| Sodium Hyaluronate | 0.0005 | 0.0005 | 0.0005 | 0.0005 |
| Ascorbic acid | | | 10.000 | |
| Water | QS | QS | QS | QS |

After the above formulations were applied, the back was subjected to UV radiation and the skin erythema was measured.

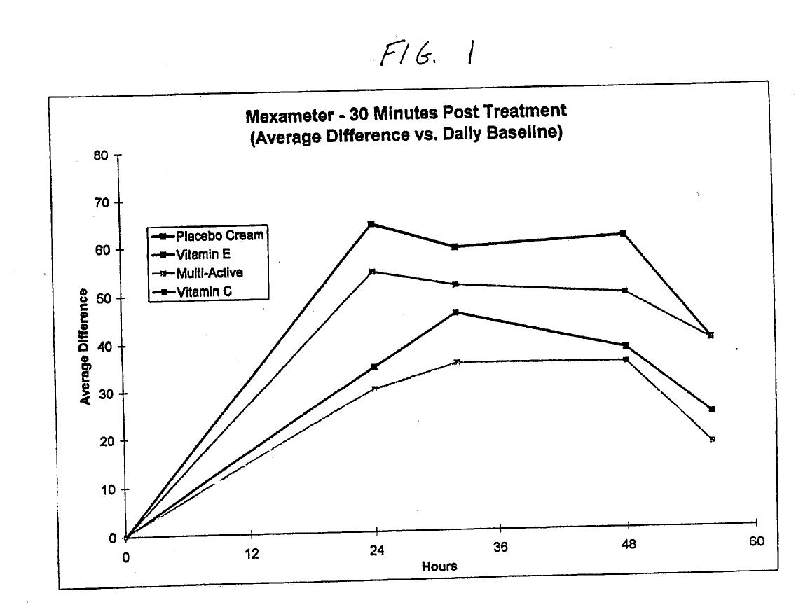

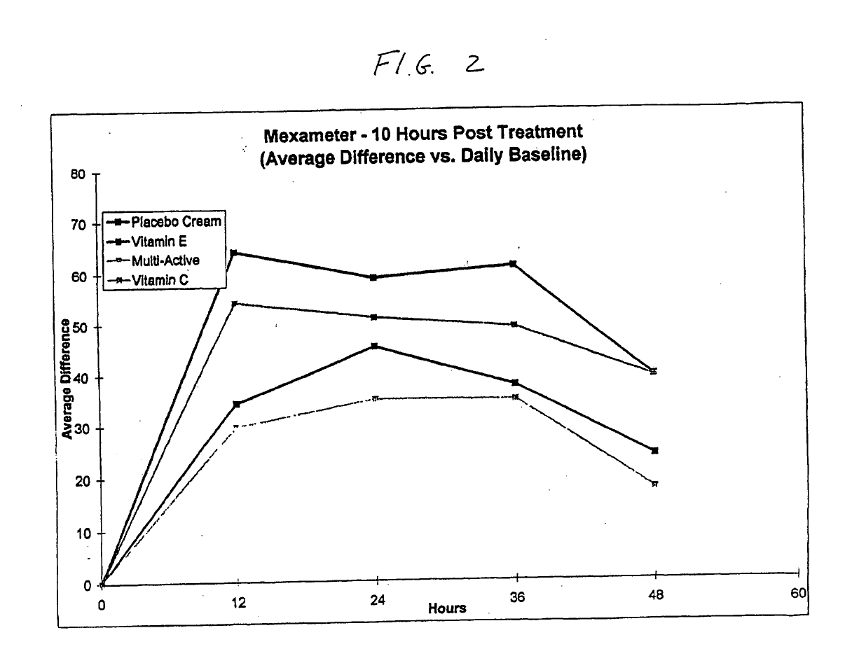

FIGS. 1 and 2 show the results.

It should be understood that a wide range of changes and modifications could be made to the embodiments described above. It is therefore intended that the foregoing description illustrates rather than limits this invention, and that it is the following claims, including all equivalents, which define this invention.

What is claimed is:

1. A composition comprising a grapefruit component, a superoxide dismutase, glutathione, tetrahydrodiferuloylmethane, a flavonoid, a grape seed component, a green tea component, tocopherol, and tocopheryl acetate.

2. A composition according to claim 1 wherein the grapefruit component is a grapefruit extract.

3. A composition according to claim 1 wherein the flavonoid is a bioflavonoid.

4. A composition according to claim 1 wherein the grape seed component is a grape seed extract.

5. A composition according to claim 1 wherein the green tea component is a green tea extract.

6. A composition according to claim 1 wherein the grape fruit component is a grapefruit extract, the flavonoid is a bioflavonoid, the grape seed component is a grape seed extract, and the green tea component is a green tea extract.

7. A composition comprising a grapefruit component, a superoxide dismutase, glutathione, a tumeric component, a flavonoid, a grape seed component, a green tea component, tocopherol, and tocopheryl acetate.

8. A composition according to claim 7 wherein the grapefruit component is a grapefruit extract.

9. A composition according to claim 7 wherein the flavonoid is a bioflavonoid.

10. A composition according to claim 7 wherein the grape seed component is a grape seed extract.

11. A composition according to claim 7 wherein the green tea component is a green tea extract.

12. A composition according to claim 7 wherein the tumeric component is a tumeric extract.

13. A composition according to claim 7 wherein the grape fruit component is a grapefruit extract, the tumeric component is a tumeric extract, the flavonoid is a bioflavonoid, the grape seed component is a grape seed extract, and the green tea component is a green tea extract.

14. A composition comprising rosemary component, lecithin, a ceramide component, a sitosterol component, glycerin, panthenol, a proline component, and a hyaluronate component.

15. A composition according to claim 14 wherein the rosemary component is a rosemary extract.

16. A composition according to claim 14 wherein the ceramide component is ceramide 3.

17. A composition according to claim 14 wherein the sitosterol component is beta-sitosterol.

18. A composition according to claim 14 wherein the hyaluronate component is sodium hyaluronate.

19. A composition according to claim 14 wherein the rosemary component is a rosemary extract, the ceramide component is ceramide 3, the sitosterol component is beta-sitosterol, and the hyaluronate component is sodium hyaluronate.

20. A composition comprising a rosemary extract and lecithin.

21. A composition comprising lecithin, ceramide 3, and beta sitosterol.

22. A composition comprising a centella asiatica component, a corn kernel component, a seaweed component, and a coenzyme component.

23. A combination according to claim 22 wherein the centella asiatica component is a centella asiatica extract.

24. A composition according to claim 22 where in the seaweed component is a seaweed extract.

25. A composition according to claim 22 wherein the coenzyme component is ubiquinone.

26. A composition according to claim 22 where the centella asiatica component is a centella asiatica extract, the seaweed component is a seaweed extract, and the coenzyme component is ubiquinone.

27. A composition comprising a soybean component, a rice component, and a sunflower seed component.

28. A composition according to claim 27 wherein the soybean component is soybean protein.

29. A composition according to claim 27 wherein the rice component is rice protein.

30. A composition according to claim 29 wherein the rice protein is hydrolyzed rice protein.

31. A composition according to claim 27 wherein the sunflower seed component is a sunflower seed extract.

32. A composition according to claim 27 wherein the soybean component is soybean protein, the rice component is hydrolyzed rice protein, and the sunflower seed component is a sunflower seed extract.

33. A method for inhibiting free radicals, reactive oxygen species, or reactive nitrogen species, comprising topically applying an effective amount of a composition comprising a grapefruit component, a superoxide dismutase, glutathione, tetrahydrodiferuloylmethane, a flavonoid, a grape seed component, a green tea component, tocopherol, and tocopheryl acetate.

34. A method for inhibiting free radicals, reactive oxygen species, or reactive nitrogen species, comprising topically applying an effective amount of a composition comprising a rosemary component, lecithin, a ceramide component, a sitosterol component, glycerin, panthenol, a proline component, and a hyaluronate component.

35. A method for inhibiting free radicals, reactive oxygen species, or reactive nitrogen species, comprising topically applying an effective amount of a composition comprising a centella asiatica component, a corn kernel component, a seaweed component, and a coenzyme component.

36. A method for inhibiting free radicals, reactive oxygen species, or reactive nitrogen species, comprising topically applying an effective amount of a composition comprising a soybean component, a rice component, and a sunflower seed component.
